(19) United States
(12) Patent Application Publication
(10) Pub. No.: US 2022/0339315 A1
(43) Pub. Date: Oct. 27, 2022

(54) ORGANOHYDROGEL FIBERS FOR SIMULTANEOUS RELEASE CONTROL OF HYDROPHILIC AND HYDROPHOBIC SUBSTANCES

(71) Applicant: North Carolina State University, Raleigh, NC (US)

(72) Inventors: Lilian Hsiao, Raleigh, NC (US); Kristine Smith, Robbinsville, NJ (US)

(21) Appl. No.: 17/724,997

(22) Filed: Apr. 20, 2022

Related U.S. Application Data
(60) Provisional application No. 63/177,452, filed on Apr. 21, 2021.

(51) Int. Cl. A61L 15/44; A61L 15/48; A61L 15/60; A61L 15/26; C08J 3/075; C08J 3/24; C08J 3/28

(52) U.S. Cl. CPC A61L 15/44; A61L 15/48; A61L 15/60; A61L 15/26; C08J 3/075; C08J 3/247; C08J 3/28; A61L 2300/414; A61L 2300/402; A61L 2300/45; A61L 2300/802; A61L 2300/602

(57) ABSTRACT

In various exemplary embodiments, the present disclosure provides organohydrogel fibers and a process for making the organohydrogel fibers. The organohydrogel fibers have a hydrophobic phase dispersed in a hydrophilic phase. The organohydrogel fibers contain at least one hydrophobic active pharmaceutical ingredient (API), and at least one hydrophilic API. The organohydrogel fibers can be formed into a non-woven or 3D printed patch and a replaceable backing can be attached to the patch to make an effective wound dressing. The wound dressing can deliver active pharmaceutical ingredients to the wound over a period of multiple days.

### CROSS-REFERENCE

This application claims priority to U.S. Provisional Application Ser. No. 63/177,452 filed on Apr. 21, 2021, which is incorporated herein by reference in its entirety.

### FIELD

The invention relates generally to organohydrogel fibers with at least one active pharmaceutical ingredient (API). The organohydrogel fibers can be used in wound and/or burn dressings to facilitate healing.

### BACKGROUND

The current standard of care for burn victims involves the use of topical creams and impermeable dressings applied to the wound sites. Some advanced dressings use hydrogels or polymers infused with silver ions for infection control. For effective infection control, these methods are combined with oral antibiotics taken as prophylaxis and analgesics for pain management. Current burn dressings require frequent replacement to remove exudate and apply topical creams. These dressing changes lead to repeated disruption of the newly formed tissue, prolonging the healing process. Furthermore, frequent dressing changes are a major source of pain and discomfort to patients and are a primary vector for wound infection. The combination with systemic oral antibiotic and analgesic medications also leads to side effects and addiction in some patients.

To reduce the economical and human costs associated with burn treatments, an advanced dressing should ideally have extended wear, provide two-way moisture control, and contain and deliver a mixture of anti-septics, anti-histamines, analgesics, bioactives, and biofilm disruptors. Furthermore, these dressings should be manufactured using extrusion methods to ensure scalability.

Two major roadblocks persist in hydrogel dressings for burn care. While there are many topical burn dressings available on the market, none have addressed these two challenges in conjunction. First, the shelf life of hydrogels containing complex mixtures of active pharmaceutical ingredients (APIs) is limited because of a strong tendency for incompatible components to de-mix, so dressings available on the market do not incorporate multiple types of API required to meet burn treatment needs. Second, commercial hydrogels are brittle and must be supported by a secondary backing, which reduces the available surface for API and bioactive delivery while adhering undesirably to wounds. Hydrogel burn dressings that address both issues suffer from expensive post-processing steps and cannot be manufactured economically at scale.

There is a need to develop dressings that offer simultaneous loading and release of a broad class of immiscible APIs. There is also a need for release kinetics for each API to be independent and to be tunable using standard manufacturing conditions (e.g., temperatures and flow rates) without the need for expensive post-processing steps. There is a need for dressings that do not adhere to fibroblasts, the cells responsible for healing wounds, making it easy to remove the dressing without causing pain. There is a need for a permeable dressing pad that is durable for multiple days and allows for moisture control at the wound. Meeting one or more of these needs is important in creating flexible and breathable burn dressings that do not need to be changed frequently, provide efficient healing of large wounds while bringing comfort to patients, and do not require new capital investments or specialized processes to manufacture.

Although the needs specific to burn wounds have been expressed, one skilled in the art will understand that organohydrogel fibers offering a simultaneous loading and release of a broad class of immiscible APIs can be used in many medical applications beyond burn dressings.

### SUMMARY OF THE INVENTION

According to an exemplary embodiment of the invention, an organohydrogel fiber comprises a hydrophobic phase dispersed within a hydrophilic phase. The organohydrogel fiber is formed from a precursor. The precursor comprises water, a gelling agent, a surfactant, a crosslinking agent, an oil, at least one hydrophobic active pharmaceutical ingredient (API), and at least one hydrophilic API. The hydrophobic phase comprises a majority of the at least one hydrophobic API and the hydrophilic phase comprises a majority of the at least one hydrophilic API.

According to another exemplary embodiment of the invention, a process for making organohydrogel fibers comprises: a) providing a precursor, b) spinning the precursor into organohydrogel fibers; c) crosslinking the gelling agent; and gathering the organohydrogel fibers. The organohydrogel fibers comprises a hydrophobic phase dispersed within a hydrophilic phase. The organohydrogel fibers are formed from a precursor. The precursor comprises water, a gelling agent, a surfactant, a crosslinking agent, an oil, at least one hydrophobic active pharmaceutical ingredient (API) and at least one hydrophilic API. The hydrophobic phase comprises a majority of the at least one hydrophobic API and the hydrophilic phase comprises a majority of the at least one hydrophilic API.

According to yet another exemplary embodiment of the invention, a wound dressing comprises a) a patch comprising organohydrogel fibers, and b) a removably attachable backing. The patch has a frontside and a backside. The removably attachable backing is attached to the patch backside and the patch frontside is fluidly connectable to a wound site. The organohydrogel fibers comprises a hydrophobic phase dispersed within a hydrophilic phase. The organohydrogel fibers are formed from a precursor. The precursor comprises water, a gelling agent, a surfactant, a crosslinking agent, an oil, at least one hydrophobic active pharmaceutical ingredient (API) and at least one hydrophilic API. The hydrophobic phase comprises a majority of the at least one hydrophobic API and the hydrophilic phase comprises a majority of the at least one hydrophilic API.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1. is a graph showing UV-vis spectroscopy (peak wavelength=290 nm) measured concentration of lidocaine normalized by its saturation concentration (12 mg/ml) for the organohydrogel fibers of Examples 1 and 2;

FIG. 2. is a graph showing UV-vis spectroscopy (peak wavelength=290 nm) measured dissolution profile of lidocaine over time for the organohydrogel fibers of Examples 1 and 2;

FIG. 3a.-3d. are pictures of in vitro micrographs showing fluorescent dermal fibroblasts that adhered to the conventional hydrogel structures of Comparative Examples 3a-3d, a) fibroblast cells adhering to noncolloidal hydrogel of Comparative Example 3a (33 wt. % PEGDA), b) F=fibroblast cells adhering to noncolloidal hydrogel of Comparative Example 3b (33 wt. % PEGDA and 0.6 wt. % bentonite), c) fibroblast cells adhering to noncolloidal hydrogel of Comparative Example 3a (33 wt. % PEGDA and 2.5 wt. % alginate), and d) fibroblast cells adhering to noncolloidal hydrogel of Comparative Example 3b (33 wt. % PEGDA, 1.25 wt. % alginate and 0.6 wt. % bentonite);

FIGS. 4a and 4b are graphs of calibration curves for (a) coumarin (C6) and (b) methylene blue (MB) dissolved in phosphate-buffered saline solution (PBS) with 0.73 wt % sodium dodecyl sulfate (SDS);

FIG. 5a-5d are graphs of C6 release profiles from crosslinked hydrogel slabs at room temperature (RT) and heated to three temperatures above the gel point temperature, T_{gel}+2° C., T_{gel}+10° C., T_{gel}+15° C., the graphs showing (a) initial timepoints fit with linear regression (dashed line) to determine slope and calculate effective diffusion coefficient, (b) linear fit for two phase diffusion in T_{gel}+10° C. and T_{gel} 15° C. samples only, (c) release profiles where dashed line is fit to principle plateau model, where T_{gel}+10° C. and T_{gel}+15° C. plots show two phase diffusion, and (d) release profiles of just RT and T_{gel}+2° C. samples with adjusted y-axis to show detail;

FIG. 6a-6c are confocal laser scanning micrographs of thermoresponsive nanoemulsion slabs, crosslinked at room temperature, T_{gel}+2° C., and T_{gel}+10° C. White is oil phase and black is aqueous phase, with scale bars representing 5 μm, showing that increasing the temperature to above the gel point causes the hydrophobic domains in the uncrosslinked hydrogels to form interconnected pores;

FIG. 7 is a confocal laser scanning micrograph of an organohydrogel fiber formed by 3D printing at a flow rate of 11 μl/s with white as the oil phase and black as the aqueous phase, scale bars represent 5 μm;

FIGS. 8a and 8b are graphs of the cumulative release of C6 from hydrogel patches printed at 11 μl/s, 17 μl/s, and 20 μl/s (a) plotted versus t^{0.5} at initial timepoints and (b) plotted versus t over the full experimental timespan; and

FIGS. 9a and 9b are graphs of cumulative release of MB from hydrogel patches printed at 11 μl/s, 17 μl/s, and 20 μl/s (a) plotted versus t^{0.5} at initial timepoints and (b) plotted versus t over the full experimental timespan.

### DETAILED DESCRIPTION

The present invention provides in an exemplary embodiment, an organohydrogel fiber comprising a hydrophobic phase dispersed within a hydrophilic phase. The organohydrogel fiber is formed from a precursor. The precursor comprises water, a gelling agent, a surfactant, a crosslinking agent, an oil, at least one hydrophobic active pharmaceutical ingredient (API), and at least one hydrophilic API. The hydrophobic phase comprises a majority of the at least one hydrophobic API and the hydrophilic phase comprises a majority of the at least one hydrophilic API.

It is to be understood that the mention of one or more method steps does not preclude the presence of additional method steps before or after the combined recited steps or intervening method steps between those steps expressly identified. Moreover, the lettering of method steps or ingredients is a conventional means for identifying discrete activities or ingredients and the recited lettering can be arranged in any sequence, unless otherwise indicated.

As used herein, the singular forms “a,” “an,” and “the” include plural referents unless the context clearly dictates otherwise. For example, “an organohydrogel fiber” can refer to one or more organohydrogel fibers. As such, the terms “a”, “an”, “one or more” and “at least one” can be used interchangeably. Also, the plural referents include the singular form unless the context clearly dictates otherwise.

As used herein, the term “and/or”, when used in a list of two or more items, means that any one of the listed items can be employed by itself, or any combination or two or more of the listed items can be employed. For example, if a composition is described as containing compounds A, B, “and/or” C, the composition may contain A alone; B alone; C alone; A and B in combination; A and C in combination; B and C in combination; or A, B, and C in combination.

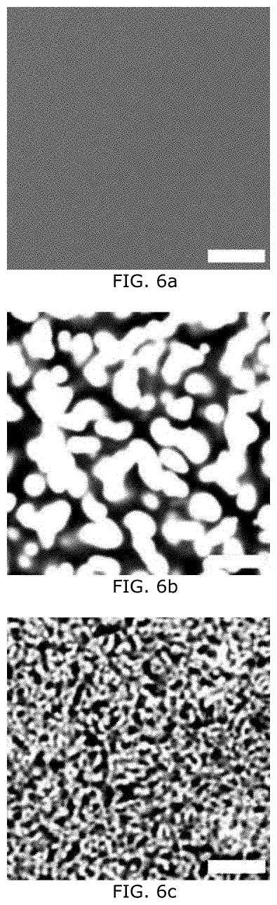

As used herein, the term “dispersed” as in “a hydrophobic phase dispersed within a hydrophilic phase” refers to discreet, unconnected areas of hydrophobic phase within the continuous hydrophilic phase and/or interconnected areas of hydrophobic phase within the continuous hydrophilic phase, for example as shown in FIGS. 6b and 6c.

As used herein, the term “precursor” refers to a two-liquid-phase mixture with an oil phase and an aqueous phase which can be processed into an organohydrogel fiber.

As used herein, the term “fiber” as in an organohydrogel “fiber” refers to the organohydrogel fiber formed by a spinning/extrusion process, whether in a traditional spinning process or by 3D printing. The precursor undergoes shear in the making of organohydrogel fiber and the amount of shear may influence the emulsion domain sizes.

As used herein, the term “hydrophobic active pharmaceutical ingredient” or “hydrophobic API”, refers to an API that preferentially partitions into the oil phase of the precursor (i.e., more than half of the “hydrophobic API” is in the oil phase of the precursor after mixing). As used herein, the term “hydrophilic active pharmaceutical ingredient” or “hydrophilic API”, refers to an API that preferentially partitions into the aqueous phase of the precursor (i.e., more than half of the “hydrophilic API” is in the aqueous phase of the precursor after mixing).

As used herein, the term “release rate” refers to the flux of the API over time. The calculation is based upon the release of the API as measured in vitro in a phosphate buffered saline solution over a time period of from 1 hour to 24 hours. The calculation and based upon the available contact area of the organohydrogen fibers (e.g., μg/cm²−hr). The available contact area is the section of the organohydrogel fibers that would be in contact with skin when used as intended.

The organohydrogel fibers are formed from a precursor. The precursor comprises water, a gelling agent, a surfactant, a crosslinking agent, an oil, at least one hydrophobic active pharmaceutical ingredient (API) and at least one hydrophilic API. In some aspects, the gelling agent comprises Poly(ethylene glycol) diacrylate (PEGDA). In some aspects, the surfactant comprises sodium dodecyl sulfate (SDS). In some aspects, the oil comprises Polydimethylsiloxane (PDMS). In some aspects, the crosslinking agent is activated by ultraviolet light and/or heat.

In some aspects, the precursor comprises 30 vol % to 90 vol % water, or 40 vol % to 85 vol % water, or 50 vol % to 80 vol % water. In some aspects, the precursor comprises 1 wt. % to 60 wt. %, or 5 wt. % to 50 wt. %, or 20 wt. % to 35 wt. % of the gelling agent. In some aspects, the precursor comprises 10 mM to 500 mM, or 30 mM to 350 mM, or 50 mM to 200 mM of the surfactant. In some aspects, the precursor comprises 0.1 wt. % to 5.0 wt. %, or 0.1 wt. % to 3.5 wt. % or 0.1 to 1.0 wt. % of the crosslinking agent. In some aspects, the precursor comprises 1 vol % to 40 vol %, or 5 vol % to 35 vol %, or 10 vol % to 30 vol % of the oil. In some aspects, the precursor comprises 0.05 wt. % to 5.0 wt. %, or 0.5 wt. % to 3.0 wt. %, or 0.7 wt. % to 2.5 wt. % of the least one hydrophobic API. In some aspects, the precursor comprises 0.05 wt. % to 5.0 wt. %, or 0.5 wt. % to 3.0 wt. %, or 0.7 wt. % to 2.5 wt. % of the least one hydrophilic API.

The precursor comprises at least one hydrophobic API and at least one hydrophilic API. In some aspects, the precursor comprises 1 to 10, or 1 to 7, or 1 to 5, or 2 to 10, or 2 to 7, or 2 to 5 hydrophobic API. In some aspects, the precursor comprises 1 to 10, or 1 to 7, or 1 to 5, or 2 to 10, or 2 to 7, or 2 to 5 hydrophilic API.

In some aspects, the at least one hydrophobic API is selected from the group consisting of analgesics, antimicrobials, antiseptics, humectants, antihistamines, biologicals, antibacterials, anti-inflammatories, biofilm disruptors, antibiotics, and/or healing agents. In some aspects, the at least one hydrophilic API is selected from the group consisting of analgesics, antimicrobials, antiseptics, humectants, antihistamines, biologicals, antibacterials, anti-inflammatories, biofilm disruptors, antibiotics, and/or healing agents.

In some aspects, the at least one hydrophobic API is selected from the group consisting of lidocaine, benzocaine (base form), fibroblast growth factor, and/or coumarin 6. In some aspects, the at least one hydrophilic API is selected from the group consisting of benzocaine, mafenide acetate, silver nitrate, Pluronic F127, propylene glycol, cetrimide, chlorhexidine, diphenhydramine HCl, bacitracin, silver ions, and/or methylene blue.

The dimensions of the organohydrogel fibers are not particularly limited. In some aspects, the organohydrogel fibers have a diameter ranging in size from 10 μm to 1,000 μm. Other non-limiting examples of the diameter size ranges are from 10 μm to 750 μm, or from 10 μm to 500 μm, or from 10 μm to 250 μm, or from 10 μm to 100 μm, or from 25 μm to 1,000 μm, or from 25 μm to 750 μm, or from 25 μm to 500 μm.

An advantage of the organohydrogel fibers is that immiscible API's can be released over time. In some aspects, a portion of the at least one hydrophobic API diffuses from the organohydrogel fibers over a period of at least 5 days, at least 10 days, or at least 14 days as measured in vitro in PBS solution. In some aspects, a portion of the at least one hydrophilic API diffuses from the organohydrogel fibers over a period of at least 5 days, at least 10 days, or at least 14 days as measured in vitro in a phosphate buffered saline solution.

The release rate for C6 from a thin slab is approximately 0.03 μg cm⁻² hr⁻¹ for RT samples from t=0.5 to 24 hours and 0.002 μg cm⁻² hr⁻¹ from t=24 to 168 hours; 0.04 μg cm⁻² hr⁻¹ and 0.004 μg cm⁻² hr⁻¹ for T_{gel}+2° C. samples, 0.13 μg cm⁻² hr⁻¹ and 0.11 μg cm⁻² hr⁻¹ for T_{gel}+10° C. samples, and 0.14 μg cm⁻² hr⁻¹ and 0.15 μg cm⁻² hr⁻¹ for T_{gel}+15° C. samples. Flux calculations are based on the contact region of the slab or patch with skin. By increasing the process temperature, we increased the release rate of the hydrophobic molecule and therefore demonstrate tunable diffusion kinetics.

The release rate for C6 from a 3D printed patch is approximately 0.00013 μg cm⁻² hr⁻¹ and 0.000016 μg cm⁻² hr⁻¹ for a flow rate of 11 μl/s, 0.00011 μg cm⁻² hr⁻¹ and 0.000012 μg cm⁻² hr⁻¹ for 17 μl/s, and 0.00008 μg cm⁻² hr⁻¹ and 0.000005 μg cm⁻² hr⁻¹ for 20 μl/s. The process temperature was T_{gel}+15° C. for these printed patches.

The release rate for MB from the same 3D printed patch is 0.00004 μg cm⁻² hr⁻¹ for the first 1 hour, and 0.0000018 μg cm⁻² hr⁻¹ for t=1 h to 120 h at a print flow rate of 11 μl/s, 0.00010 μg cm⁻² hr⁻¹ and 0.00002 μg cm⁻² hr⁻¹ at 17 μl/s, and 0.00016 μg cm⁻² hr⁻¹ and 0.0000028 μg cm⁻² hr⁻¹ at 20 μl/s. The process temperature was T_{gel}+15° C. for these printed patches. The difference between C6 and MB release rates is that increasing the shear rate causes C6 to be released more gradually while MB to be released much more rapidly, demonstrating independent tuning of the dual release kinetics of immiscible APIs.

In some aspects, the present embodiment allows for different release rates for the at least one hydrophobic API and the at least one hydrophilic API. In some aspects, the release rate of one of the at least one hydrophobic API differs from the release rate of one of the at least one hydrophilic API by at least 5%, as measured in vitro in a phosphate buffered saline solution. Other non-limiting examples of differences in release rate include at least 10% or at least 20% or at least 50% or at least 100%.

In another exemplary embodiment of the invention, a process for making organohydrogel fibers comprises a) providing a precursor, b) spinning the precursor into organohydrogel fibers; c) crosslinking the at least one gelling agent; and gathering the organohydrogel fibers. The organohydrogel fibers comprises a hydrophobic phase dispersed within a hydrophilic phase. The organohydrogel fibers are formed from a precursor. The precursor comprises water, a gelling agent, a surfactant, a crosslinking agent, an oil, at least one hydrophobic active pharmaceutical ingredient (API) and at least one hydrophilic API. The hydrophobic phase comprises a majority of the at least one hydrophobic API and the hydrophilic phase comprises a majority of the at least one hydrophilic API.

It is to be understood that the various aspects of the organohydrogel fiber described above, including the precursor composition, the cross-linker, the number of hydrophobic and hydrophilic APIs, the types of hydrophobic and hydrophilic APIs, specific examples of hydrophobic and hydrophilic APIs, time of diffusion of hydrophobic and hydrophilic APIs, relative release rates of hydrophobic and hydrophilic APIs, and fiber dimensions, apply to the present embodiment as well.

In some aspects, the precursor is produced by a process comprising: a) combining the water, the gelling agent, the surfactant, and optionally the at least one hydrophilic API together to make a first portion, b) combining the oil and optionally the at least one hydrophobic API together to make a second portion, c) combining the first portion and the second portion to make a two-phase mixture, wherein the two-phase mixture comprises a precursor hydrophobic phase and a precursor hydrophilic phase, d) optionally adding the at least one hydrophobic API and/or the at least one hydrophilic API to the two-phase mixture, and e) adding the cross-linking agent. The at least one hydrophobic API is added at step b) and/or step d), and the at least one hydrophilic API is added at step a) and/or step d).

In some aspects, spinning the precursor is done by 3D printing the precursor. In some aspects, wet spinning is used to make the organohydrogel fibers. In some aspects, electrospinning is used to make the organohydrogel fibers. In some aspects, the shear rate that the precursor undergoes in spinning can be optimized for a desired release rate of the at least one hydrophobic API and/or the release rate of the at least one hydrophilic API. In some aspects, the shear rate during spinning ranges from 0.05 s⁻¹ to 0.3 s⁻¹. Other non-limiting examples of shear rate ranges during spinning include from 0.08 s⁻′ to 0.25 s⁻′, or from 0.10 s⁻¹ to 0.25 s⁻¹, from 0.1 s⁻¹ to 0.2 s⁻¹.

In some aspects, step c) crosslinking is initiated by ultraviolet light and/or heat.

Step d) gathering the organhydrogel fibers is not particularly limited and can be done by means well known to those skilled in the art. When 3-D printing is used for the spinning, gathering the fibers can be as simple as removing a patch or other object from the substrate onto which the fibers have been printed.

In yet another exemplary embodiment of the invention, a wound dressing comprises a) a patch comprising organohydrogel fibers, and b) a removably attachable backing. The patch has a frontside and a backside. The removably attachable backing is attached to the patch backside, and the patch frontside is fluidly connectable to a wound site. The organohydrogel fibers comprises a hydrophobic phase dispersed within a hydrophilic phase. The organohydrogel fibers are formed from a precursor. The precursor comprises water, a gelling agent, a surfactant, a crosslinking agent, an oil, at least one hydrophobic active pharmaceutical ingredient (API) and at least one hydrophilic API. The hydrophobic phase comprises a majority of the at least one hydrophobic API and the hydrophilic phase comprises a majority of the at least one hydrophilic API.

It is to be understood that the various aspects of the organohydrogel fibers described above, including the precursor composition, the cross-linker, the number of hydrophobic and hydrophilic APIs, the types of hydrophobic and hydrophilic APIs, specific examples of hydrophobic and hydrophilic APIs, time of diffusion of hydrophobic and hydrophilic APIs, relative release rates of hydrophobic and hydrophilic APIs, and fiber dimensions, apply to the present embodiment as well. It is also to be understood that they organhydrogel fibers for the wound dressing can be made by a process using any of the aspects described above.

In some aspects, the patch is selected from the group comprising a non-woven pad, a 3D-printed patch, or a molded patch comprising the organhydrogel fibers. In some aspects the patch is permeable. In some aspects the patch is breathable. In some aspects, patch remains intact after up to 3 changes of the removably attachable backing. Other non-limiting examples of the patch remaining intact include after up to 5 changes, or up to 10 changes, or up to 14 changes of the removably attachable backing.

In some aspects, a first portion the at least one hydrophobic API diffuses from the patch over a period of at least 5 days, or at least 10 days, or at least 14 days, and/or a second portion of the at least one hydrophilic API diffuses from the patch over a period of at least 5 days, or at least 10 days, or at least 14 days, as measured in vitro in a phosphate buffered saline solution. In some aspects, a release rate of one of the at least one hydrophobic API differs from the release rate of one of the at least one hydrophilic API by at least 5%, or at least 10%, or at least 20%, as measured in vitro in a phosphate buffered saline solution.

The wound dressing may be used to treat burns and other skin injuries. A non-limiting set of example active pharmaceuticals (APIs) for treating burns is given in Table 1. The target dosage shows typical concentration of the API in current creams or ointment applied to burns.

TABLE 1
List of APIs of interest to burn care

| Function | Molecule | Property | Target Dosage |
|---|---|---|---|
| Analgesic | Lidocaine | Hydrophobic | 2 wt % |
| Analgesic | Benzocaine (base) | Hydrophobic | 2 wt % |
| Antimicrobial | Silver sulfadiazine | Hydrophilic | 1 wt % |
| Antimicrobial | Mafenide acetate | Hydrophilic | 1 wt % |
| Antimicrobial | Silver nitrate | Hydrophilic | 0.5 wt % |
| Antiseptic | Pluronic F127 | Amphiphilic | 19 wt % |
| Humectant | Propylene glycol | Hydrophilic | 3 wt % |
| Antiseptic | Cetrimide | Amphiphilic | 0.1 wt % |
| Antiseptic | Chlorhexidine | Hydrophilic | 4 wt % |
| Antihistamine | Diphenhydramine HCl | Hydrophilic | 2 wt % |
| Biologic | Fibroblast growth | Hydrophobic | 1 μg/cm² |
| | factor | | |

### EXAMPLES

The formulation for thermoresponsive nanoemulsions includes poly(dimethyl siloxane) (PDMS, viscosity=5 cP) as the oil phase, sodium dodecyl sulfate (SDS, ≥99.0%, dust-free pellets) as the surfactant, poly(ethylene glycol) diacrylate (PEGDA, Mn=700 g/mol) telechelic polymer, and deionized water as the continuous phase. The alginate was Alginic acid sodium salt from brown algae, Sigma A2033-500G. Coumarin 6 (C6) and methylene blue (MB) were loaded into the oil and aqueous phases, respectively, for diffusion kinetic studies. Hydrophobic dye, PKH26 (λex/λem=551/567 nm), was used for confocal laser scanning microscopy (CLSM) imaging and photoinitiator, 2-hydroxy-2-methylpropiophenone (Darocur) was used for crosslinking polymer. Nanoclay (hydrophilic bentonite) was added to select 3D printing studies to improve structural longevity prior to photocrosslinking. Diffusion media (DM) for Examples 5, 7, and 8 was a solution of 0.73 wt % SDS in phosphate-buffered saline (lx PBS). All chemicals were purchased from Sigma-Aldrich and used without further purification.

### Example 1

50 ml of precursor was prepared by pouring 8.56 mL deionized water into a beaker, then adding 20 mL of 600 mM sodium dodecyl sulfate (SDS) and 10.95 mL Poly(ethylene glycol) diacrylate (PEGDA). The precursor was set onto a stir plate, a magnetic stir bar was added, and the stir rate was set at 750 RPM. 0.93 g of alginate was added slowly to the mixture. The beaker was covered and left on the stir plate at 750 RPM for 4-5 days. Polydimethylsiloxane (PDMS) was saturated with lidocaine at a composition of 12 mg lidocaine/mL PDMS. 10 mL of the lidocaine/PDMS solution was slowly added to the beaker, the beaker was covered, and the beaker remained on the stir plate at 750 RPM for 2-3 days. The precursor composition was 33 vol. % PEGDA, 2.5 wt. % Alginate, 300 mM SDS and 20 vol. % PDMS.

After a total mixing time of one week, the precursor was aliquoted into 8 mL portions for ultrasonication. A water bath for cooling was filled with an ice slurry and 8 mL of the precursor was placed in a 20 ml clear glass dram vial with a small magnetic stirrer. The entire sample was submerged in the ice slurry. The sonication probe was lowered about one third of the way down into the precursor volume and the stir plate was set at 750 RPM. The sonicator was set for 15 minutes (active); Amplitude at 35%; Pulse 2 seconds on and 3 seconds off and started. After one minutes, and every three minutes thereafter, the sample was unloaded and shaken vigorously for 15 seconds or vortexed for 3 seconds. Upon completion of the sonication, 1 vol % of darocur (photoinitiator) was added to the precursor, the precursor was vortexed and stored at 4° C. until further use. The crosslinked gel was soaked in 1M CaCl₂) for 24 hours following extrusion.

The precursor was loaded into a syringe (Luer-Lok tip, BD) at ambient temperature (T=20° C.) and at a volume of 3 mL. The filled syringe was placed within a custom-built temperature-controlled syringe jacket. Thermogelation was induced inside the jacket with water flowing around the syringe at 22° C. The precursor was equilibrated for 20-30 min. By use of a syringe pump, the gelled precursor was extruded through an 18-gauge (inner diameter=0.84 mm) needle that was connected to a transparent fluidic channel of equivalent inner diameter. The fluidic channel is constructed from cross-linked PDMS (Sylgard 184, Corning), which was used to prevent adhesion of the cured hydrogel fiber to the walls due to its high oxygen permeability. Furthermore, the PDMS is transparent and allows UV light to pass through the entire width of the channel. Once flow was applied, UV light was used to illuminate the PDMS channel at an intensity of 8 mW/cm². This intensity was measured at the opposite side of the channel block in which the UV light was applied. The PEGDA in the gelling precursor was covalently cross-linked by the UV light during flow through the transparent channel, resulting in solid fibers. The fibers were collected in deionized (DI) water at the outlet. Fibers containing alginate were collected in 1M CaCl solution for secondary crosslinking.

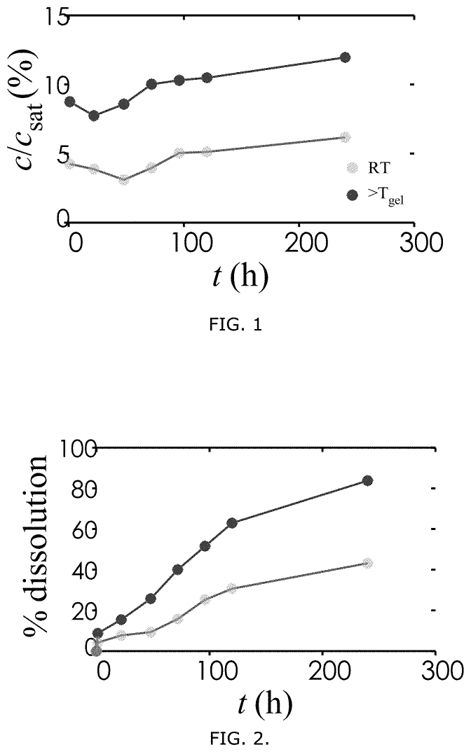

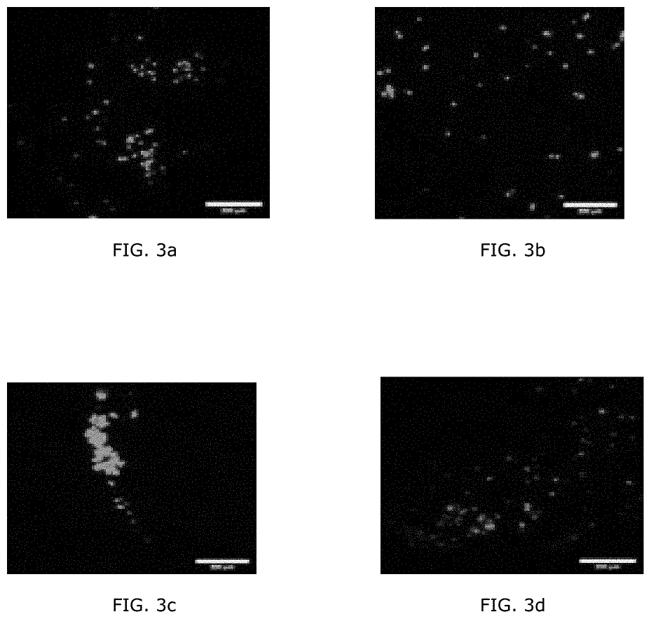

Diffusion of the lidocaine out of the organohydrogel fiber of Example 1 was measured as follows. The fiber was immersed by 1.5 mL PDMS in a dish. Each day, 1.0 mL of PDMS was removed from the dish for sampling and 1.0 mL of fresh PDMS was added to the dish. The sample was subject to a UV absorbance scan from 190-400 nm. The result was compared to the UV absorbance curve of known lidocaine concentrations in PDMS. FIG. 1 is graph showing UV-vis spectrometry (peak wavelength=290 nm) measured concentration of lidocaine normalized by its saturation concentration (12 mg/ml) over time. FIG. 2 shows the percent dissolution, or the percent of the lidocaine in the organohydrogel fiber that has diffused into the solution, over time.

### Example 2

Some of the precursor prepared for Example 1 was formed into a fiber as done in Example 1 except that thermogelation was induced inside the jacket with water flowing around the syringe at 35° C. The diffusion of lidocaine was measured as in Example 1 and is shown in FIG. 1. and FIG. 2.

The Example 1 and Example 2 are hydrogels formed at room temperature and hydrogels formed above the gelation point, respectively. The main difference is that above the gel point, large hydrophobic domains formed which facilitate the release of the hydrophobic lidocaine. FIG. 1. shows a graph of UV-vis spectroscopy (peak wavelength=290 nm) measured concentration of lidocaine normalized by its saturation concentration (12 mg/ml) for the organohydrogel fibers of Examples 1 and 2. Example 2, which was formed above the gel point, shows a higher release of the hydrophobic lidocaine. FIG. 2. shows a UV-vis spectroscopy (peak wavelength=290 nm) graph showing the dissolution profile of lidocaine over time of Examples 1 and 2.

### Comparative Examples 3a-3d

Non-colloidal gels were made similar to Example 1, without the addition of PMDS or lidocaine. All Comparative Examples 3a-3d had 33 wt. % PEDGA as did Examples 1 and 2. Comparative Example 3b also contained 0.6 wt. % bentonite, Comparative Example 3c also contained 2.5 wt. % alginate, and Comparative Example 3d contained 1.25 wt. % alginate and 0.6 wt. % bentonite. Comparative Examples 3a-3d were cured under UV light for 3-5 minutes. 40 uL 0.1M CaCl₂) was dropped onto Comparative Examples 3c and 3d. These gels were covered and stored at 4° C. until subject to cell culture.

The nanoemulsion from Example 1 and the gels from Comparative Examples 3a-3d were subject to fibroblasts, the cells responsible for healing wounds, culture. The cells were imaged before and after rinsing each sample with sterile PBS. The cells were imaged on the confocal using the following settings.

Vybrant DiI: excitation=549 nm, emission=565 nm,

LIVE: excitation=488 nm, emission=515 nm

DEAD: excitation=570 nm, emission=602 nm

Objective—10×

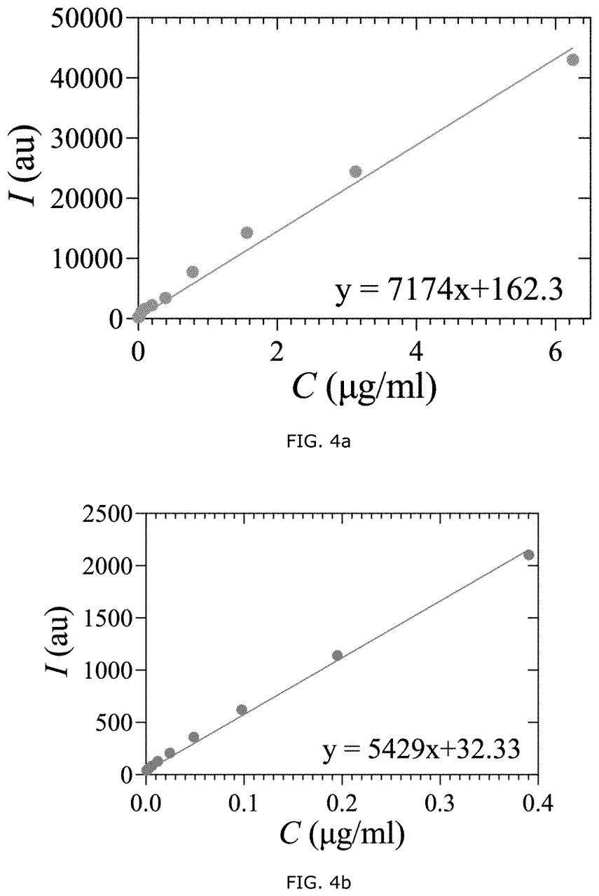

FIGS. 3a.-3d. show fluorescent images of cells after the PBS rinse for Comparative Examples 3a.-3d., respectively. FIG. 3a. shows cells with normal morphology taken after a gentle rinse with PBS, indicating that the cells are attached to the gel and not floating in media. FIG. 3c. shows that the presence of alginate appears to have cells spreading, clumping together, or moving into pores of the gel. Comparing FIGS. 3a. to 3b. shows that incorporation of bentonite at this low concentration does not yield significant difference in cell growth.

No cells were found on the gel from Example 1. As the fibroblasts did not adhere to the gel of inventive Example 1, the hydrogel has promise for being removed from wounds without cell adherence and the corresponding pain and disruption to healing.

### Example 4

8 ml batches of nanoemulsions were prepared as follows. PDMS oil (ϕ=0.2) was added dropwise to an aqueous solution containing PEGDA (33 vol %) and SDS (200 mM) mixed at 550 rpm in a 50 ml beaker. Agitation rate was increased to 750 rpm and the emulsion was mixed for 30 min before transfer to a glass vial for ultrasonication using a Cole-Palmer 750-watt ultrasonic homogenizer. During ultrasonication, an ice bath was used to prevent overheating of the sample. Mixing continued at 750 rpm throughout the ultrasonication process along with inversion and vortex mixing at 1, 3, 5, 7 and 9 minutes. The ultrasonication time was 12 min (30 min total) alternating between 2 s on and 3 s off to prevent overheating the sample. Ultrasonication amplitude was 35% and frequency was 20 kHz. Following ultrasonication, the nanoemulsion was filtered by a 1 μm nylon filter (Whatman Anotop) and stored at 5° C.

For nanoemulsions containing Coumarin 6 (C6), enough solid C6 was dissolved in PDMS oil to allow for saturation at 0.1 mg C6/ml PDMS. The solution was filtered to remove any undissolved C6. For nanoemulsions containing methylene blue (MB), MB was added to the aqueous solution containing PEGDA (33 vol %) and SDS (200 mM) until the concentration reached 0.1 mg MB/ml aqueous solution.

Nanoemulsion size was analyzed by Dynamic Light Scattering (DLS). Analysis was performed by diluting the sample to ϕ=0.002 in 33 vol % PEGDA solution. The diluted sample was loaded into a Malvern Zetasizer and the droplet size was measured as 2a=33 nm f 30%.

The gel point temperature, T_{gel}, was measured for each batch of nanoemulsions with a cone and plate rheometer setup (1°, 50 cm, DHR-2, TA Instruments) and a small amplitude oscillatory shear procedure. The sample was probed at a frequency of ω=20 rad/s and strain of y=0.05% while temperature was increased at 1° C./min from 20° C. to 60° C. The temperature where the storage modulus (G′) surpassed the loss modulus (G″) is the gel point temperature since this point is where the nanoemulsion is more solid-like than liquid like. The gel point temperatures for the batches fell within 39±3° C. and temperature setpoints were adjusted for each experiment based on the measured gel point for each nanoemulsion batch.

### Example 5

Hydrogel slabs were prepared from nanoemulsions prepared in Example 4, made with C6. 4 ml of nanoemulsion with C6 was transferred to a small vial and spiked with 1 vol % Darocur. The vial was mixed by vortexing and sonication and then stored overnight at 5° C. to allow bubble dissipation. The nanoemulsion was then transferred to a 4.95 cm diameter Pyrex dish, where it was gelled and crosslinked using a hot plate under a UV light. Each nanoemulsion dish was heated at the setpoint temperature for 10 min, at which time, the UV lamp was turned on to crosslink the gel while heating continued for 5 min. The gelation setpoint temperature was either room temperature (RT), T_{gel}+2° C., T_{gel}+10° C., or T_{gel}+15° C. Following crosslinking, each gel was rinsed with 2 ml of water and the circular slab was cut with a blade into three 15 mm×15 mm square gel slabs. Each gel slab was transferred to a diffusion cell at t=0.

Diffusion cells were modeled off USP Apparatus 5 and designed to allow for gentle mixing but prevent physical damage to the gel slabs due to contact with the stir bar. The diffusion vessel was a 20 ml vial without the cap. Each vessel contained a stir bar and was filled with 8 ml of diffusion media (DM): PBS (3 mM sodium phosphate, 150 mM sodium chloride, 1.05 mM potassium phosphate) with 0.73 wt % SDS. A 25 mm diameter nylon membrane (0.45 μm, Sigma-Aldrich) was submerged in DM but suspended above the stir bar using nylon string which was secured to the upper lip of the vial using a rubber band. Each diffusion slab was placed on top of the nylon membrane and each cell was covered in parafilm to prevent evaporation during diffusion studies. Finally, diffusion experiments were covered in a large beaker and aluminum foil to prevent light exposure.

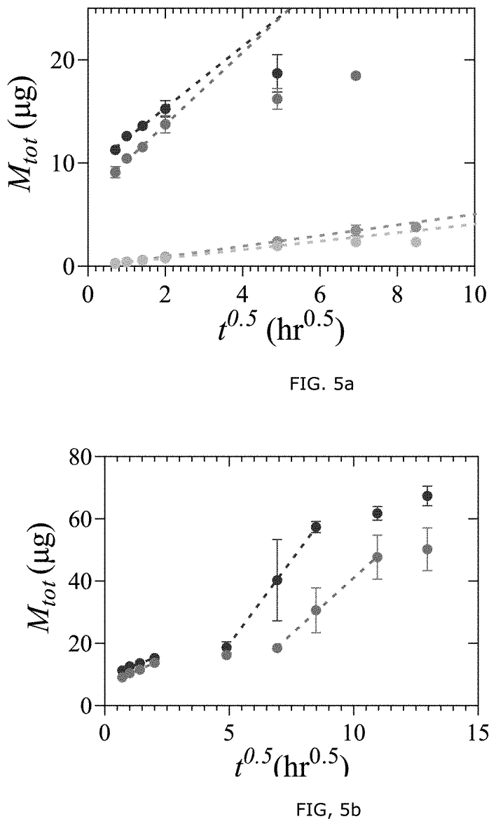

At each diffusion time point, the DM within the cell was mixed by aspirating and expelling 1 ml three times. Next, 1 ml of sample was filtered through a nylon syringe filter (0.45 μm, Thermo Scientific) into a 2 ml amber vial. The 1 ml sample was replaced with 1 ml of fresh DM to maintain constant volume and sink conditions within the cell. After sampling, diffusion cells were covered with parafilm and foil to prevent evaporation and light exposure. Fluorescence measurements were taken of the samples and compared to the calibration curve at various concentrations of C6 in the DM (FIG. 4a).

A Tecan microplate reader was used to measure fluorescence and determine concentration of C6 molecules. Calibration curves were generated for each solute in the diffusion media by 2:1 serial dilutions in a Corning 96 well flat bottom plate. The calibration curve for C6 is shown in FIG. 4a. For C6 analysis, the excitation wavelength was 485 nm, and emission wavelength was 535 nm. An equation was fitted to the linear part of the calibration curve and used to calculate concentration for diffusion samples. No samples were higher than the linear region of the curve, and therefore no sample dilutions were necessary.

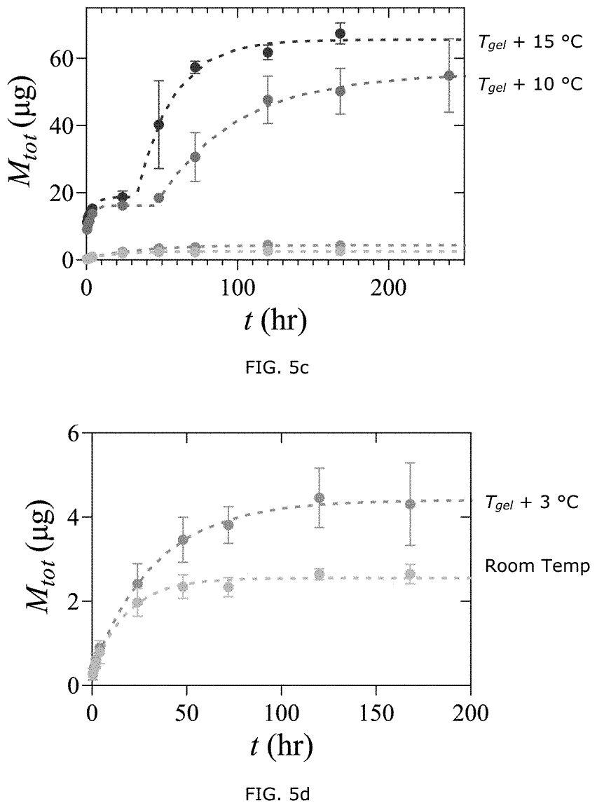

C6 release profiles from hydrogel slabs molded at room temperature (RT), T_{gel}+2° C., T_{gel}+10° C., and T_{gel}+15° C. are given in FIG. 5 FIG. 5(a) shows initial timepoints fitted with linear regression (dashed line) to determine slope and calculate effective diffusion coefficient. FIG. 5(b) shows a linear fit for two phase diffusion in T_{gel}+10° C. and T_{gel}+15° C. samples only. FIG. 5(c) shows release profiles where the dashed line is fit to the principle plateau model. T_{gel}+10° C. and T_{gel}+15° C. plots show two phase diffusion. FIG. 5(d) shows the release profiles of just room temperature and T_{gel}+2° C. samples with adjusted y-axis to show detail.

### Example 6

Nanoemulsion samples from Example 4 made with C6, were doped with 1 vol % Darocur and 1 vol % PKH26. The mixture was mixed thoroughly using a vortex mixer, then placed in a bath sonicator and allowed to rest such that bubbles could dissipate. 200 μl of the sample was aliquoted into an 8-well chambered coverglass (#1.5, Nunc™ Lab-Tek™ II). The sample was then heated to the set point temperature (Room temperature, T_{gel}+2° C., T_{gel}+10° C.) on a hotplate for 10 min and then crosslinked using a UV lamp (9.0 mW/cm², 254 nm) for 5 min while heating continued.

Confocal laser scanning microscopy (CLSM) images were captured using a Leica TCS SP8 to analyze the internal microstructure of nanoemulsion gel slabs created at different temperatures and flow rates. The CLSM was equipped with a 63× oil immersion objective (numerical aperture=1.3) and laser emitting at 552 nm. A few drops of DI water were added on top of each sample to prevent drying during imaging. The decreasing size of emulsions with increasing temperature set point can be seen in FIG. 6(a)-6(c).

The local volume fraction of the oil phase and tortuosity of the colloidal network define the effective diffusion coefficient, D_{eff}. In Example 5, slab thickness (L) and initial loaded mass of C6 (M0) were kept constant and only the internal microstructure was varied, and therefore D_{eff}.

From Example 5, we observe that increasing process temperature decreases oil domain size and increases D_{eff}. At short time periods, the square of the slopes in FIG. 5a are 0.17 μg²/hr, 0.27 μg²/hr, 12.2 μg²/hr and 8.9 μg²/hr for RT, T_{gel}+2° C., T_{gel}+10° C. and T_{gel}+15° C. respectively, where slope squared is proportional to D_{eff}. From Example 6, images of samples synthesized just above the gel point temperature reveal large oil phase domains and similarly large aqueous domains (FIG. 6a). Molecules in the oil phase must navigate around large regions where they are insoluble. This is analogous to a roadmap with few roads and large regions that cannot be driven through, such as state parks, farmland, and mountains. The path to get from A to B is longer than if there were dense and interconnected roadways such as in a city with an intersection every block. Microstructures observed at high temperatures are more homogeneous and interconnected. This structure allows for a more direct route for solutes to diffuse against the concentration gradient and out of the gel (FIG. 6).

In the two high temperature slab samples, diffusivity increases after 48 hours, resulting in an apparent two-phase release profile (FIG. 5c). After 48 hours, the squared slope of Mₜₒₜ vs t^{0.5} increase to 52.1 μg²/hr, and 116.0 μg²/hr for T_{gel}+10° C. and T_{gel}+15° C., therefore indicating D_{eff} increases 430% and 1300% compared to D_{eff} at the initial time points. Without being bound by any theory, a potential mechanism is hydrolysis and therefore degradation of PEGDA over time. Visual observation of the gel slabs following 10 days of soaking showed limited bulk degradation; all gel slabs were still intact. The diffusion cells contained remnants of gel pulp starting around day 2 or 3 which suggest partial degradation; however, pulp was observed in all temperature samples while only the high temperature setpoint experiments showed signs of two-phase release.

### Example 7

Gel patches were created from the nanoemulsions (Example 4 with C6 added) using a Cellink BioX 3D printer fitted with a syringe pump printhead and a photocuring toolhead. Darocur (1 vol %) and nanoclay (3 wt %) were added into nanoemulsion followed by vortex mixing, sonication, and settling for bubble dissipation. The nanoemulsion was then loaded into a 3 ml BD syringe with a 0.25″ blunt 22G needle. The filled syringe was inserted into the syringe pump printhead and heated to T_{gel}+15° C. for 20 minutes. The print stage was also heated to T_{gel}+15° C. to prevent the nanoemulsion from cooling following printing but prior to photocrosslinking. The geometry of the gel patch was written using g-code. The geometry was a 15 mm×15 mm patch with rectilinear pattern infill. The size was based on the size of the 20 ml vial diameter (˜25 mm). The rectilinear pattern is commonly used in 3D printing of hydrogels for templating tissues with isotropic mechanical characteristics such as skin, fascia, and cartilage. The g-code included a line unassociated with the patch used to prime the needle and expel nanoemulsion not adequately heated by the syringe pump tool. Printing was performed at three flow rates: Q=11 μl/s, 17 μl/s and 20 μl/s, with a retract volume of 6 μl. The flow rates correspond to average shear rates of 0.082 s⁻¹, 0.13 s⁻¹, and 0.15 s⁻¹, respectively. For each flow rate, the translation speed of the printer was adjusted to maintain constant volume for each patch (0.60 μl/mm). Print velocities were 18.3 mm/s, 28.3 mm/s, and 33.3 mm/s respectively. The printing substrate was a glass slide, which was heated by the printing stage. Two UV modules emitting at 365 nm and 405 nm were turned on to promote photocrosslinking during the printing process. When printing was complete, the photocuring toolhead (365 nm) continued crosslinking for 10 s and 6.5 cm above the gel patch. Following printing, each gel patch was transferred to a diffusion cell at t=0. Diffusion measurements were conducted as described in Example 5 with the gel patch resting on the nylon membrane in place of the slab.

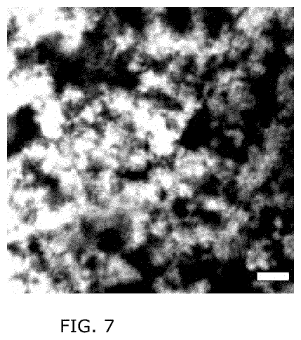

FIG. 7 shows a confocal laser scanning micrograph of organohydrogel fibers produced at the lower flow rate, 11 μl/s, corresponding to a shear rate of 0.082 s⁻¹.

### Example 8

Example 7 was repeated except gel patches were created from the nanoemulsions Example 4 (with MB added). Darocur (1 vol %) and nanoclay (3 wt %) were added into nanoemulsion. Following printing, each gel patch was transferred to a diffusion cell at t=0. Diffusion measurements were conducted as described in Example 5 with the gel patch resting on the nylon membrane in place of the slab.

A Tecan microplate reader was used to measure fluorescence and determine concentration of MB molecules. Calibration curves were generated for each solute in the diffusion media by 2:1 serial dilutions in a Corning 96 well flat bottom plate. Calibration curve for MB is shown in FIG. 4b. For MB analysis, the excitation wavelength was 530 nm, and the emission wavelength was 680 nm. An equation was fitted to the linear part of the calibration curve and used to calculate concentration for diffusion samples. No samples were higher than the linear region of the curve and therefore no sample dilutions were necessary

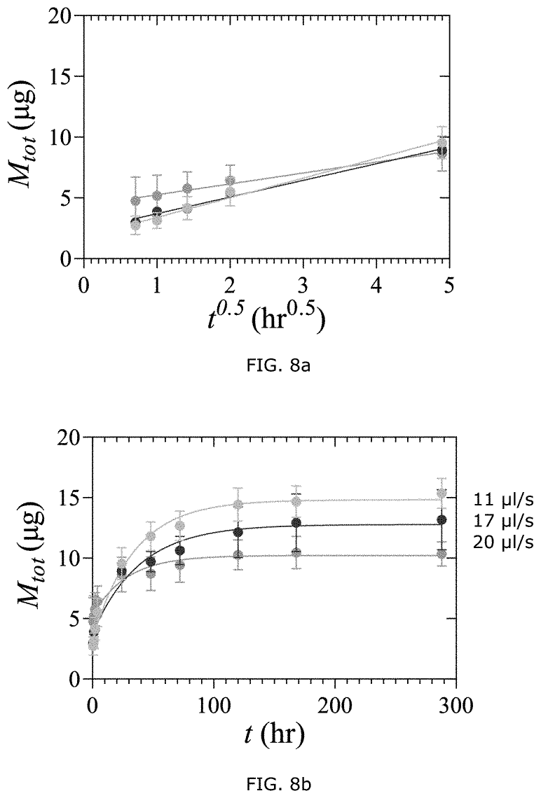

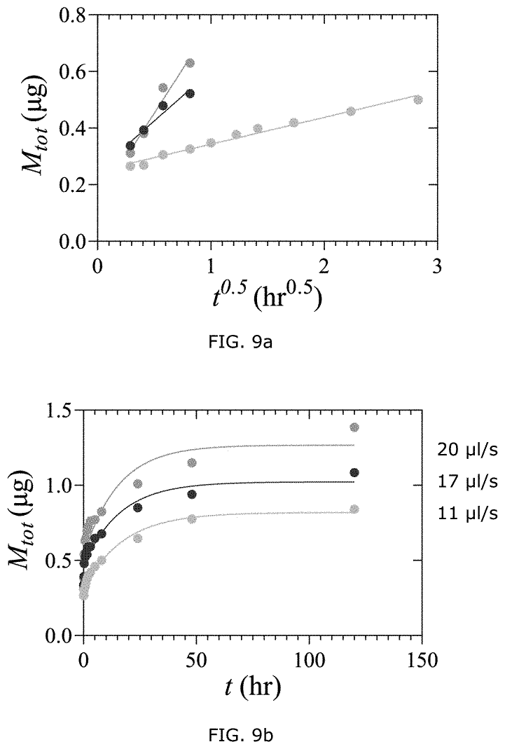

The impact of process shear on molecule release and diffusivity in colloidal gels was evaluated by 3D printing gel patches at different extrusion rates and determining the release profiles of C6 (Example 7) and MB (Example 8) loaded into the oil and aqueous phases, respectively. For these experiments, process temperature, needle size, patch geometry and volume were kept constant. FIG. 8 and FIG. 9 show the release profile of C6 and MB from gel batches produced at 11 μl/s, 17 μl/s, and 20 μl/s. The trend for each molecule is opposite. For C6 samples, the effective diffusion coefficient decreased with increasing extrusion rate. At short time periods, D_{eff} for 11 μl/s, 17 μl/s, and 20 μl/s is proportional to the squared slope of Mₜₒₜ vs t^{0.5}: 2.6 μg²/hr, 1.9 μg²/hr, and 0.80 μg²/hr. Conversely for MB samples, effective diffusion coefficient increased with increasing extrusion rate where D_{eff} for 11 μl/s, 17 μl/s, and 20 μl/s is proportional to 0.01 μg²/hr, 0.12 μg²/hr, and 0.39 μg²/hr. Without being bound by any theory, we propose the same release mechanisms in the 3D printed samples as in the slabs. Hydrophobic molecules must travel by pathways created by oil phase domains, therefore navigating around hydrophilic regions, and hydrophilic molecules must diffuse along aqueous phase pathways around hydrophobic regions. MB diffusion is additionally slowed by the PEGDA hydrogel mesh in the aqueous phase. Although all samples were printed at the highest temperature condition, T_{gel}+15° C., we do not see the same two-phase diffusion that was observed in the slab samples. Without being bound by any theory, this may be because shearing the colloids at high temperatures, representative of strong attractive interactions, leads to the formation of large dense colloidal domains and heterogeneous voids.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An organohydrogel fiber comprising a hydrophobic phase dispersed within a hydrophilic phase, wherein the organohydrogel fiber is formed from a precursor, and wherein the precursor comprises water, a gelling agent, a surfactant, a crosslinking agent, an oil, at least one hydrophobic active pharmaceutical ingredient (API), and at least one hydrophilic API,
wherein the hydrophobic phase comprises a majority of the at least one hydrophobic API, and
wherein the hydrophilic phase comprises a majority of the at least one hydrophilic API.

2. The organohydrogel fiber of claim 1, wherein the gelling agent comprises Poly(ethylene glycol) diacrylate (PEGDA), the surfactant comprises sodium dodecyl sulfate (SDS), and the oil comprises Polydimethylsiloxane (PDMS).

3. The organohydrogel fiber of claim 1, wherein the crosslinking agent is activated by ultraviolet light and/or heat.

4. The organohydrogel fiber of claim 1, wherein the organohydrogel fiber comprises 30 vol % to 90 vol % water, 1 wt. % to 60 wt. % of the gelling agent, 10 mM to 500 mM of the surfactant; 0.1 wt. % to 5.0 wt. % of the crosslinking agent, 1 vol % to 40 vol % of the oil, 0.05 wt. % to 5.0 wt. % of the least one hydrophobic API, and 0.05 wt. % to 5.0 wt. % of the least one hydrophilic API.

5. The organohydrogel fiber of claim 1, wherein the precursor comprises 1 to 5 of the least one hydrophobic API and 1 to 5 of the least one hydrophilic API.

6. The organohydrogel fiber of claim 1, wherein the at least one hydrophobic API is selected from the group consisting of analgesics, antimicrobials, antiseptics, humectants, antihistamines, biologicals, antibacterials, anti-inflammatories, biofilm disruptors, antibiotics, and/or healing agents; and/or wherein the at least one hydrophilic API is selected from the group consisting of analgesics, antimicrobials, antiseptics, humectants, antihistamines, biologicals, antibacterials, anti-inflammatories, biofilm disruptors, antibiotics, and/or healing agents.

7. The organohydrogel fiber of claim 1, wherein the at least one hydrophobic API is selected from the group consisting of lidocaine, benzocaine (base form), fibroblast growth factor, and/or coumarin 6; and/or wherein the at least one hydrophilic API is selected from the group consisting of benzocaine, mafenide acetate, silver nitrate, Pluronic F127, propylene glycol, cetrimide, chlorhexidine, diphenhydramine (HCL), bacitracin, silver ions, and/or methylene blue.

8. The organohydrogel fiber of claim 1, wherein the organohydrogel fiber has a diameter ranging in size from 10 μm to 100 μm.

9. The organohydrogel fiber of claim 1, wherein a first portion of at least one of the at least one hydrophobic API diffuses from the organohydrogel fiber over a period of at least 5 days, and/or a second portion of at least one of the at least one hydrophilic API diffuses from the organohydrogel fiber over a period of at least 5 days, as measured in vitro in a phosphate buffered saline solution.

10. The organohydrogel fiber of claim 1, wherein a first release rate of one of the at least one hydrophobic API differs from a second release rate of one of the at least one hydrophilic API by at least 5%, wherein each of the release rates is measured in vitro in a phosphate buffered saline solution over a time period of from 0.5 hour to 24 hours and is based upon the available contact area of the organohydrogen fibers.

11. A process for making organohydrogel fibers, the process comprising:
a) providing a precursor;
b) spinning the precursor into the organohydrogel fiber;
c) crosslinking the at least one gelling agent; and
d) gathering the organohydrogel fibers,
wherein the organohydrogel fibers comprise a hydrophobic phase dispersed within a hydrophilic phase, wherein the organohydrogel fiber is formed from a precursor, and
wherein the precursor comprises water, a gelling agent, a surfactant, a crosslinking agent, an oil, at least one hydrophobic API, and at least one hydrophilic API,
wherein the hydrophobic phase comprises a majority of the at least one hydrophobic API, and
wherein the hydrophilic phase comprises a majority of the at least one hydrophilic API.

12. The process of claim 11, wherein the precursor is produced by a process comprising:
a) combining the water, the gelling agent, the surfactant, and optionally the at least one hydrophilic API together to make a first portion;
b) combining the oil and optionally the at least one hydrophobic API together to make a second portion;
c) combining the first portion and the second portion to make a two-phase mixture, wherein the two-phase mixture comprises a precursor hydrophobic phase and a precursor hydrophilic phase;
d) optionally adding the at least one hydrophobic API and/or the at least one hydrophilic AP1 to the two-phase mixture; and
e) adding the cross-linking agent,
wherein the at least one hydrophobic API is added at step b) and/or step d), and the at least one hydrophilic API is added at step a) and/or step d).

13. The process of claim for 11, wherein the step b) spinning is selected from the group consisting of 3D-printing, wet spinning, and electric spinning, and wherein the shear rate during spinning ranges from 0.05 s⁻¹ to 0.3 s⁻¹.

14. The process of claim 11, wherein the step c) crosslinking is initiated by ultraviolet light and/or heat.

15. A wound dressing comprising:
a) a patch comprising organohydrogel fibers; and
b) a removably attachable backing,
wherein the patch has a frontside and a backside, wherein the removably attachable backing is attached to the patch backside, and the patch frontside is fluidly connectable to a wound site,
wherein the organohydrogel fibers comprise a hydrophobic phase dispersed within a hydrophilic phase, wherein the organohydrogel fibers are formed from a precursor, and
wherein the precursor comprises water, a gelling agent, a surfactant, a crosslinking agent, an oil, at least one hydrophobic API, and at least one hydrophilic API,
wherein the hydrophobic phase comprises a majority of the at least one hydrophobic API, and
wherein the hydrophilic phase comprises a majority of the at least one hydrophilic API.

16. The wound dressing of claim 15, wherein the patch is selected from the group consisting of a non-woven pad, a 3D-printed patch, or a molded patch comprising the organhydrogel fibers.

17. The wound dressing of claim 15, wherein the patch is permeable and/or breathable.

18. The wound dressing of claim 15, wherein the patch remains intact after up to 3 changes of the removably attachable backing.

19. The wound dressing of claim 15, wherein a first portion of the at least one hydrophobic API diffuses from the patch over a period of at least 5 days, and/or a second portion of the at least one hydrophilic API diffuses from the patch over a period of at least 5 days, as measured in vitro in a phosphate buffered saline solution.

20. The wound dressing of claim 15, wherein a first release rate of one of the at least one hydrophobic API differs from a second release rate of one of the at least one hydrophilic API by at least 5%, as measured in vitro in a phosphate buffered saline solution.
